(19) United States
(12) Patent Application Publication
(10) Pub. No.: US 2009/0233914 A1
(43) Pub. Date: Sep. 17, 2009

(54) Composition for treatment of nail infections

(75) Inventors: Nicholas G. Loebel, Redmond, WA (US); Fergus R. McKenzie, Kinross (GB); Cale Street, Edmonds, WA (US); Roger Andersen, Ladysmith (CA); Robert Scott, Auburn, WA (US)

Correspondence Address: DOBRUSIN & THENNISCH PC, 29 W LAWRENCE ST, SUITE 210, PONTIAC, MI, 48342, US

(73) Assignee: Ondine International, Ltd.

(21) Appl. No.: 12/075,361

(22) Filed: Mar. 11, 2008

(51) Int. Cl. A61K 31/5415; A61K 31/045; A61K 31/19; A61P 31/10; A61K 31/125

(52) U.S. Cl. 514/224.8; 514/729; 514/557; 514/675

(57) ABSTRACT

The present invention includes a composition for treating fungal infections, particularly onychomycosis. The composition includes a photosensitizer, an effective amount of antifungal agent, and a pharmaceutically acceptable delivery system.

### CLAIM OF BENEFIT OF FILING DATE

This application claims the benefit of U.S. Provisional Application Ser. No. 60/893,722 titled: “Therapy and Composition for Nail Infections” filed on Mar. 8, 2007.

### CROSS-REFERENCE TO RELATED APPLICATIONS

The present application is related to concurrently filed, commonly owned, copending U.S. patent application entitled “Therapy and Device for Treatment of Nail Infections” (Attorney Docket No. 1248-076) which is hereby incorporated by reference.

### FIELD OF THE INVENTION

The present invention relates to composition, therapy (i.e., method) and device for treating fungal infections. Specifically, the invention described herein is particularly useful for the treatment of onychomycosis.

### BACKGROUND OF THE INVENTION

Both humans and animals can be afflicted with microorganisms that invade beneath the nail, claw or hoof resulting in a condition which causes discoloration, thickening, brittleness, pain, and ultimately loss of the affected nail, claw or hoof.

This condition, known an onychomycosis or Tinea unguium (ringworm of the nails), is caused primarily by members of a group of parasitic fungi known as Trichophyton rubrum or Trichophyton mentagrophytes, and occasionally by Aspergillus fumigatus. Epidermophyton floccosum, Microsporum canis, Microsporum gypsum and other organisms may also be causative agents of onychomycosis. Onychomycosis is particularly prevalent in humans, affecting 15-20% of the population.

Known oral treatments for onchomycosis have to date been marginally effective and expensive. Traditionally, onychomycosis has been treated with an oral medicine known as Griseofulvin®, which is largely ineffectual and has undesired side effects. Other treatments used to combat onychomycosis include Lamisil® (terbinafine), which is taken once a day for 90 days resulting in nail clearing in 70-80% of patients for one year but is expensive. Another medicine, Sporanox® (itraconazole), is taken twice a day for one week each month over the course of three to four months and is also expensive. In addition, the side effects of itraconazole are somewhat severe and the remission is 60-80%. Fluconazole may also be used to treat onychomycosis; however, it also has severe side effects. Given the poor cure rate, undesirable side effects and high costs associated with existing treatments, a significant need exists to effect cost effective treatments for onychomycosis.

Topical preparations of known antifungal agents for treatment of onychomycosis exist. However, since the pathogenic fungus resides in the nail bed, effective topical treatments must be able to penetrate the affected nail in order to avoid surgical removal of the nail. Use of chemical permeation enhancers has been a common approach for enhancing trans-nail delivery of antifungal agents. See e.g., U.S. Pat. Nos. 6,042,845; 6,159,977; 6,224,887 and 6,391,879. These nail penetration enhancers has been incorporated with an antifungal agent to treat onychomycosis. Conventional topical antifungal agents, even via a trans-nail delivery system, still require multiple applications over a period of time.

Thus, there is a need for effective topical antifungal compositions and therapy which are safe and effective in treating onychomycosis with limited side effects. In particular there is a need for effective, safe, faster, and less expensive topical treatments for onychomycosis.

Accordingly, it is a primary object of the invention to provide topical compositions and therapy which are particularly useful against fungal infections, particularly onychomycosis. Other features and advantages of the present invention will be apparent to those skilled in the art from the following detailed description and claims.

### SUMMARY OF THE INVENTION

The present invention is directed to a composition, a therapy (i.e., method), and a light delivery device for treating fungal infections, particularly onychomycosis. The antifungal composition of the present invention includes a photosensitizer, an effective amount of antifungal agent, and a pharmaceutically acceptable delivery system, preferably for delivering the photosensitizer and the antifungal agent through the nail to the site of treatment (“locus”). It is preferred that the antifungal agent is one or more of a compound selected from the group consisting of (-) menthol, menthone, menthyl salicylate, (-)(1R) menthyl acetate, (-)(1R) menthyl chloride and menthyloxyacetic acetic acid. It is further preferred that the antifungal agent further comprises camphor.

The therapy of the present invention includes: applying the antifungal composition to a locus (either directly or indirectly by applying the composition on the nail beneath which the locus is situated), allowing sufficient penetration time, and irradiating the locus with a light source at a wavelength absorbed by the photosensitizer so as to destroy microbes at the locus. Since light can penetrate through the nail to the locus, the irradiating step is achieved by irradiating either directly on the locus (with nail removed), or indirectly through the nail with a light source at a wavelength absorbed by the photosensitizer so as to destroy microbes at the locus.

The therapy of the present invention may optionally include (i) reducing the thickness of the nail and/or (ii) creating micro channels from top of the nail to bottom of the nail via ablation means prior to the application of the antifungal composition.

The light delivery of the present invention includes (i) a housing adapted to cover the nail beneath which the locus is situated; (ii) a light source, (iii) an energy source in power communication to the light source; and (iv) a controller that controls amount and/or duration of light to be applied to the locus by the light source.

### BRIEF DESCRIPTION OF THE DRAWINGS

The features and inventive aspects of the present invention will become more apparent upon reading the following detailed description, claims, and drawings, of which the following is a brief description:

FIG. 1 is a pictorial prospective view of an exemplary embodiment of a device according to the present invention;

FIG. 2 is another pictorial prospective view of the device shown in FIG. 1 used for treatment on a user's toe;

FIG. 3 is a graph showing the amount of viable fungal colonies of Trichophyton rubrum after treatment relative to control in four different experiments described below in Example I;

FIG. 4 is a photograph of an agar plate that has been incubated for 72 hours and subject to the experiment described below in Example II;

FIG. 5 is a photograph of an agar plate that has been incubated for 96 hours and subject to the experiment described below in Example II;

FIG. 6 is a photograph of an agar plate that has been incubated for 144 hours and subject to the experiment described below in Example II; and

FIG. 7 is a photograph of an agar plate that has been incubated for about 9 days and subject to the experiment described below in Example II.

### DETAILED DESCRIPTION OF THE INVENTION

Photodynamic therapy has been known to be effective in killing microbes including fungus. Topical preparations of antifungal agents can also be used to treat nail infections. For example, in vitro microbiological tests of a topical antifungal agent comprising one or more of the following active compounds; (-) menthol, or a menthol derivative or analog, e.g., menthone, menthyl salicylate, (-) (1R) menthyl acetate, (-) (1R) menthyl chloride, and menthyloxyacetic acid, and preferably further comprising camphor, showed that it is effective in the topical treatment of fungal infections, particularly onychomycosis, as well as dermatophytic fungi. The antifungal agents' effectiveness depends, in part, upon the ability of the antifungal agents to reach the locus. Delivery of the antifungal agents can be achieved by removing the nail and applying the antifungal agent directly onto the locus, or using a pharmaceutically acceptable nail penetration enhancer to deliver the antifungal agent through the nail without removal of the nail itself.

The present invention uses photodynamic therapy in combination with an antifungal agent to provide a synergistic effect in that photodynamic therapy delivers immediate killing of microbes and disinfects the locus while the antifungal agent provides long term killing of fungi at the locus. With the use of a pharmaceutically acceptable nail penetration enhancer, the present invention also provides a composition and a therapy whereby nail removal is not required for the effective killing of microbes.

I. Definitions

The following terms are intended to have the following general meanings as they are used herein.

1. Microbes: any and all disease-related microbes such as virus, fungus, and bacteria including Gram-negative organisms, Gram-positive organisms or the like.

2. Light: light at any wavelengths that can be absorbed by a photosensitizing composition. Such wavelengths include wavelengths selected from the continuous electromagnetic spectrum such as ultraviolet (“UV”), visible, the infrared (near, mid and far), etc. The wavelengths are generally preferably between about 160 nm to 1600 nm, more preferably between 400 nm to 800 nm, most preferably between about 500 nm to 850 nm although the wavelengths may vary depending upon the particular photosensitizing compound used and the light intensity. The light may be produced by any suitable art-disclosed light emitting devices such as lasers, light emitting diodes (“LEDs”), incandescent sources, fluorescent sources, or the like.

3. Locus: any area where anti-microbial treatment is desired around a nail and/or dermis (e.g., nail bed or the like).

4. Nail: any nail of an animal (e.g., fingernail, toenail, hoof, claw, or the like).

5. Photosensitizer: any suitable art-disclosed photosensitizer. Arianor steel blue, toluidine blue O, crystal violet, methylene blue and its derivatives, azure blue cert, azure B chloride, azure 2, azure A chloride, azure B tetrafluoroborate, thionin, azure A eosinate, azure B eosinate, azure mix sicc., azure II eosinate, haematoporphyrin HCI, haematoporphyrin ester, aluminium disulphonated phthalocyanine are examples of suitable photosensitizers. Porphyrins, pyrroles, tetrapyrrolic compounds, expanded pyrrolic macrocycles, and their respective derivatives are further examples of suitable photosensitizers. Photofrin® manufactured by QLT Photo Therapeutics Inc., Vancouver, B. C., Canada is yet another example of a suitable photosensitizer. Other exemplary photosensitizers may be found in U.S. Pat. Nos. 5,611,793 and 6,693,093. U.S. Pat. No. 6,693,093 is hereby incorporated by reference. The photosensitizers mentioned above are examples are not intended to limit the scope of the present invention in any way.

6. Animal: any and all animals including but not limited to humans, cows, horses, sheep, etc.

7. Dermatophytic fungal infection: an infection of the dermis or nails by a fungus. Such fungi include, but are not limited to, Trichophyton rubrum, Trichophyton mentagrophytes, Epidermophyton floccosum, Aspergillus fumigatus, and Candida albicans. In particular, such an infection can be called “onychomycosis,” which is a general term referring to the infection of the nail by any fungal species.

8. Menthol derivative or analog: a molecule that shares structural and functional features in common with menthol, and which may be prepared by chemical treatment of menthol. For purposes of the present invention, a menthol derivative or analog has antifungal activity. Examples of derivatives and analogs include, but are not limited to menthone, menthyl salicylate, menthyl acetate, menthyl chloride, and menthoxyacetic acid.

9. Pharmaceutically acceptable: molecule entities and compositions that are physiologically tolerable and do not typically produce an allergic or similar untoward reaction, such as gastric upset, dizziness and the like, when administered to a human. Preferably, as used herein, the term “pharmaceutically acceptable” means approved by a regulatory agency of the Federal or a state government, or listed in the U.S. Pharmacopeia or other generally recognized pharmacopeia for use in animals, and more particularly in humans.

10. Carrier: a diluent, adjuvant, excipient, or vehicle with which the compound is administered. Such pharmaceutical carriers can be sterile liquids, such as water and oils, including those of petroleum, animal, vegetable or synthetic origin, such as peanut oil, soybean oil, mineral oil, sesame oil and the like. Carriers can also be multi-step preparations designed to modify the nail protein conformation or composition to increase permeability before applying the antifungal agent. Water or aqueous solution saline solutions and aqueous dextrose and glycerol solutions are preferably employed as carriers, particularly for injectable solutions. A pharmaceutically acceptable carrier preferably enhances delivery of the active agent (menthol or menthol derivative or analog) to the nail bed. Suitable pharmaceutical carriers are described in “Remington's Pharmaceutical Sciences” by E. W. Martin, 17^{th} Edition.

11. Effective amount: an amount sufficient to reduce by at least about 15 percent, preferably by at least 50 percent, more preferably by at least 90 percent, and most preferably prevent, a clinically significant deficit in the activity, function and response of the host. Alternatively, an effective amount is sufficient to cause an improvement in a clinically significant condition in the host.

II. Antifungal Composition

The antifungal composition of the present invention contains a photosensitizer, an effective amount of an antifungal agent, and a pharmaceutically acceptable delivery system.

The photosensitizer can be any suitable art-disclosed photosensitizer. A preferred exemplary photosensitizer is methylene blue or its derivatives. Depending on the desired application, the composition may comprise a plurality of photosensitizers. The amount or concentration of the photosensitizer(s) may vary depending upon the desired application, the particular photosensitizer(s) used, and the target microbes to be destroyed. In one embodiment of the present invention, the concentration of the photosensitizer(s) in the antifungal composition is preferably from about 0.00001% to about 50% w/v, more preferably from about 0.0001% to about 25% w/v, still more preferably from about 0.001% to about 10% w/v, and most preferably from about 0.01% to about 1% w/v.

The antifungal agent can be any suitable art-disclosed antifungal agent. For example, polyenes (e.g., Natamycin, Nystatin, or the like), allylamines (e.g., Naftifine, Terbinafine, or the like), imidazoles (e.g., Bifonazole, Chlotrimazole, Econazole, Fenticonazole, Ketocanazole, Miconazole, Oxiconazole, or the like), triazoles (e.g., Fluconazole, Itraconazole, Terconazole, or the like), tolnaftate, ciclopirox, morpholines (e.g., amorolfine or the like), griseofulvin, or the like, and a combination thereof. See also The Merck Index under the headings of “Antifungal (Antibiotic)” and “Antifungal (Synthetic)” in the Therapeutic Category and Biological Activity Index.

A preferred exemplary antifungal agent includes one or more of the following active compounds: (-) menthol, a menthol derivative or analog (e.g., menthone, menthyl salicylate), (-) (1R) menthyl acetate, (-) (1R) menthyl chloride, and menthyloxyacetic acid. It is also preferred that this antifungal agent includes camphor. The amount of such active compounds in the antifungal composition may range from about 1% to about 50%, about 2% to about 25%, preferably about 2% to about 10%, more preferably about 3% to about 10%, most preferably about 4% to about 5% by weight of the total antifungal composition. Additionally, camphor may be added in an amount of from about 1% to about 15%, preferably about 2% to about 11%, more preferably about 2% to about 5% by weight of the total antifungal composition.

The pharmaceutically acceptable delivery system includes an art-disclosed pharmaceutically acceptable carrier. Petroleum hydrocarbons selected as the carrier are preferably high molecular weight hydrocarbons, with a melting point above body temperature (37° C.). For example, petroleum or petroleum jelly may be employed as suitable carriers for the active ingredient(s). As both menthol and camphor are poorly soluble in water, petrolatum has been found to be a suitable vehicle to use.

For those patients who prefer a non-sticky/greasy water based gel as the carrier, the composition may be prepared in a creme formulation. Creme formulations are widely used, industry standard, buffered formulations, typically used for agents which are soluble in alcohol and poorly soluble in water. Such cremes may contain cetyl alcohol, cetyl palmitate, copolyol, EDTA, glycerin, H₂O, imidazole-urea, isopropyl palmitate, methyl paraben, PEG-100 stearate, sodium hydroxide, turpentine, stearic acid, or stearyl alcohol.

Cremes which include lotions, salves, and the like, are well known in the art. A preferred creme formulation comprises ingredients selected from a C₈-C₂₀ long chain alcohol, a C₁₀-C₄₀ long chain ester, C₈-C₂₀ long chain carboxylic acid, a copolyol, EDTA, glycerin, water, imidazole urea; methyl paraben, polyethylene glycol 100 stearate, sodium hydroxide and turpentine.

A preferred alcoholic-based gel carrier contains a C1-C9 alkyl alcohol, preferably ethanol, present in about 15% to about 50% of the total antifungal composition. The carrier also preferably includes a gel forming agent, preferably either hydroxypropyl cellulose or carboxymethylcellulose present in a concentration of about 1% to 5% by weight of the total antifungal composition. The alcohol-based gel antifungal composition can be brought to 100% by the addition of water and brought to neutral pH (e.g., about pH 7) by the addition of sodium hydroxide. Preferably the alcohol used in the antifungal composition should be water free.

The pharmaceutically acceptable delivery system may optionally include a suitable art-disclosed pharmaceutically acceptable nail penetration enhancer. Examples of such nail penetration enhancer are provided in U.S. Pat. Nos. 6,042,845; 6,159,977; 6,224,887 and 6,391,879. These patents are hereby incorporated by reference. The nail penetration enhancer allows the delivery of the photosensitizer and the antifungal agent through the nail to the locus without nail removal. Nail removal is likely to cause discomfort to the patient, and therefore, eliminating the need for nail removal is generally desirable in the nail infection therapy.

The antifungal composition may optionally comprise addition components such as anti-inflammatory agents, buffers, salts for adjusting the tonicity of the solution, antioxidants, preservatives (e.g., propylene glycol, methyl paraben, or the like).

An preferred exemplary embodiment of the antifungal composition comprises the photosensitizer; the pharmaceutically acceptable nail penetration enhancer; about 0.5% to about 2% (w/w) carboxymethylcellulose; about 30% (w/w) ethanol; about 15% propylene glycol or methyl paraben; about 4% (w/w) menthol; and about 2% (w/w) camphor; Water is then added to bring the total to 100% and the solution neutralized to about pH 7 by the addition of sodium hydroxide.

The antifungal composition may be packaged in an appropriate container. The antifungal compositions may be supplied in bottles with brush applicators or applicator tipped bottles or glass rod applicator bottles.

In another exemplary embodiment of the present invention, the antifungal composition does not include the antifungal agent, but instead is comprised of the photosensitizer and the pharmaceutically acceptable delivery system described above. The pharmaceutically acceptable delivery system preferably includes the pharmaceutically acceptable nail penetration enhancer discussed above. This embodiment of the antifungal composition can be used alone or in conjunction with the antifungal agent in a secondary and separate application to treat nail infections. It is preferred that the antifungal agent is combined with the pharmaceutically acceptable delivery system, especially one that includes the pharmaceutically acceptable nail penetration enhancer.

III. Therapy for Dermatophytic Fungal Infections

The present invention provides a therapy for dermatophytic fungal infections comprising applying the antifungal composition to a locus and irradiating the locus with a light source at a wavelength absorbed by the photosensitizer so as to destroy microbes at the locus. Generally, to avoid discomfort to the patient, it is preferred that the nail above the locus is not removed. Nevertheless, it is contemplated and within the scope of the present invention to include the optional step of removing the nail before the application of the antifungal composition and the irradiation step.

If the nail is removed as part of the therapy, the antifungal composition does not need a pharmaceutically acceptable nail penetration enhancer. Also, in the nail is removed as part of the therapy, it is also within the scope of the present invention to first apply the photosensitizer to a locus; irradiate the locus with a light source at a wavelength absorbed by the photosensitizer so as to destroy microbes at the locus, and then apply a composition comprising the antifungal agent and the pharmaceutically acceptable carrier.

An exemplary protocol for the therapy for treatment of onychomycosis is as follows:
1. Apply the antifungal composition of the present invention to the nail beneath which the locus is situated;
2. Irradiating the locus with a light source at a wavelength absorbed by the photosensitizer of the antifungal composition so as to destroy microbes at the locus (this irradiating step also does not require the removal of the nail because the irradiation will penetrate the nail thereby allowing irradiation of the locus); and
3. Repeated steps 1-2 described above during each 24 hours period over a predetermined treatment period (e.g., days, weeks, or months).
   This exemplary protocol and the therapy of the present invention may optionally include washing and drying the nail beneath which the locus is situated before application of the composition.

If the antifungal composition does not include the antifungal agent, then the exemplary protocol can be changed to the following:
1. Apply the antifungal composition of the present invention to the nail beneath which the locus is situated;
2. Irradiating the locus with a light source at a wavelength absorbed by the photosensitizer of the antifungal composition so as to destroy microbes at the locus (this irradiating step also does not require the removal of the nail because the irradiation will penetrate the nail thereby allowing irradiation of the locus); and
3. Apply the antifungal agent wherein the antifungal agent is preferably combined with the pharmaceutically acceptable delivery system, especially one that includes the pharmaceutically acceptable nail penetration enhancer.
4. Repeat steps 1-3 described above during each 24 hours period over a predetermined treatment period (e.g., days, weeks, or months).

As discussed in Examples IV and V below, the therapy of the present invention may also optionally include (i) reducing the thickness of the nail and/or (ii) creating micro-channels from top of the nail to bottom of the nail via art-disclosed ablation means prior to the application of the composition. For example, the ablation means can either mechanically or electromagnetically ablate micron-thin sections of the nail until the desired thickness is reached. It is preferred that the desired thickness is a thickness that causes little or no discomfort to the patient.

The light applied during the irradiating step of the therapy can be supplied by a single light emitting device or a plurality of light emitting devices. Any suitable art-disclosed light emitting device(s) such as lasers, light emitting diodes (“LEDs”), incandescent sources, fluorescent sources, or the like may be used to provide the wavelength(s) that can be absorbed by the photosensitizer. Lasers include any art-disclosed lasers such as diode lasers, gas lasers, fibers lasers or diode pumped solid state laser or the like. LEDs include any art-disclosed LEDs such as semiconductor LEDs, organic LEDS or a combination thereof. Fluorescent sources include any art-disclosed fluorescent sources such as fluorescent tubes, LED pumped fluorescent devices, cold cathode fluorescent panels or the like.

The light applied during the irradiating step of the therapy provides the wavelength(s) that can be absorbed by the photosensitizer. Such wavelength(s) include wavelengths selected from the continuous electromagnetic spectrum such as ultra violet (“UV”), visible, the infrared (near, mid and far), etc. The wavelengths are generally preferably between about 160 nm to 1600 nm, more preferably between 400 nm to 900 nm, most preferably between about 500 nm to 850 nm although the wavelengths may vary depending upon the particular photosensitizer used and the light intensity.

The time required for the irradiating step of the therapy may vary depending on the existing conditions (e.g., the microbes, the photosensitizer, the light source, etc.). Once the photosensitizer has been delivered effectively through the nail to the site of infection, it is preferred that the photosensitizer is left in contact with the locus for a period of time to enable the microbes located near or at the locus to take up some of the photosensitizer and become sensitive to it. For example, the light applied during the irradiating step of the therapy can be applied by a high energy power for short durations. A suitable duration will generally be from about 1 second to about 30 minutes, preferably about 30 seconds to about 10 minutes, more preferably about 1 minute to about 5 minutes and most preferably about 3 minutes. It is also possible and within the scope of the present invention for the light applied during the irradiating step of the therapy to be applied by a lower energy power for much longer durations (e.g., more than about 30 minutes to hours).

The therapy of the present invention is preferably applied to the locus every 1-2 days (i) for at least about one week for a milder infection and (ii) for about one or more months to clear a more advanced infection, or where the entire nail is involved. Therapeutic effectiveness is observed by a reversal of nail deterioration or pain, and by an improvement in nail appearance. The therapy of the present invention can also be used to treat re-infection, should that occur.

IV. Light Delivery Device for Therapy for Dermatophytic Fungal Infections

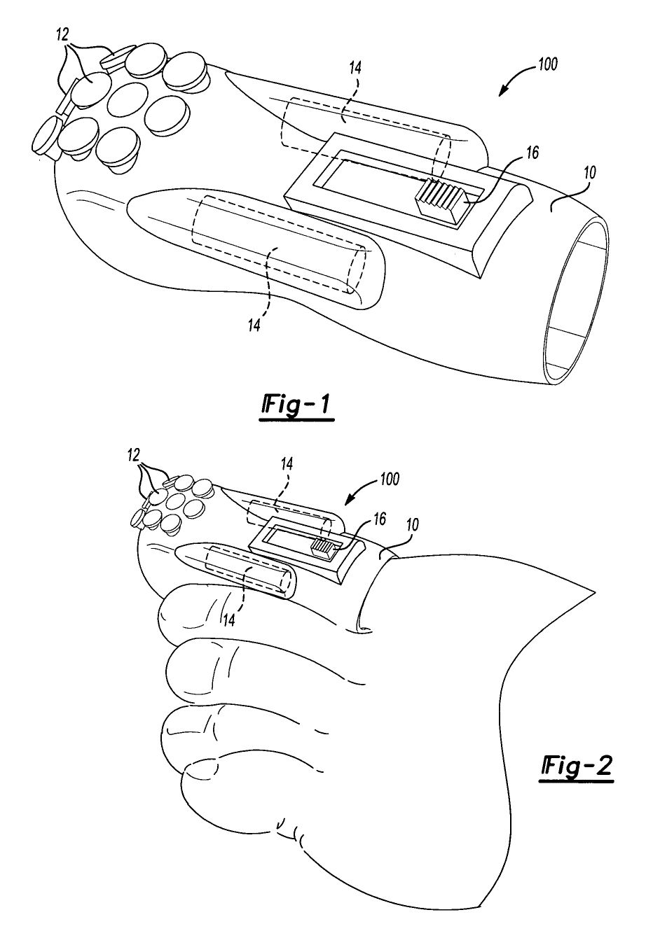

If long duration light exposure is desired during the irradiating step of the therapy, the present invention provides a light delivery device 100 adapted for such use. Referring to FIG. 1, the device 100 includes a housing 10 adapted to cover the nail beneath which the locus is situated. The housing is optionally designed to cover not just the nail but also to accept a substantial portion of the toe itself as shown in FIGS. 1 and 2. The device 100 further includes a light source 12. The light source 12 can be any suitable art-disclosed light emitting device(s) such as lasers, LEDs, incandescent sources, fluorescent sources, or the like may be used to provide the wavelength(s) that can be absorbed by the photosensitizer. In one exemplary embodiment, the light source 12 is an array of LEDs as showed in FIG. 1. In another exemplary embodiment, the light source 12 is an array of optical fibers powered by a laser. The device 100 further includes an energy source 14 in power communication with the light source 12 and is adapted to provide power to the light source 12. The energy source 14 can be DC and/or AC. The housing 10 can optionally be adapted to contain the energy source 14 (e.g., batteries or the like) as shown in FIG. 1. Alternatively, the energy source 14 can be located outside of the housing 10 but is in power communication with the light source 12 via cable(s). The device 100 further includes a controller 16 that controls the amount (including duration) of the light that is applied to the locus.

The present invention is not being limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

It is further to be understood that all numerical values are approximate and are provided for description only.

Patents, patent applications, and publications cited throughout this application are incorporated herein by reference in their entireties.

The following examples provided in accordance to the present invention are for illustrative purpose only and are not intended as being exhaustive or limiting of the invention.

### EXAMPLE I

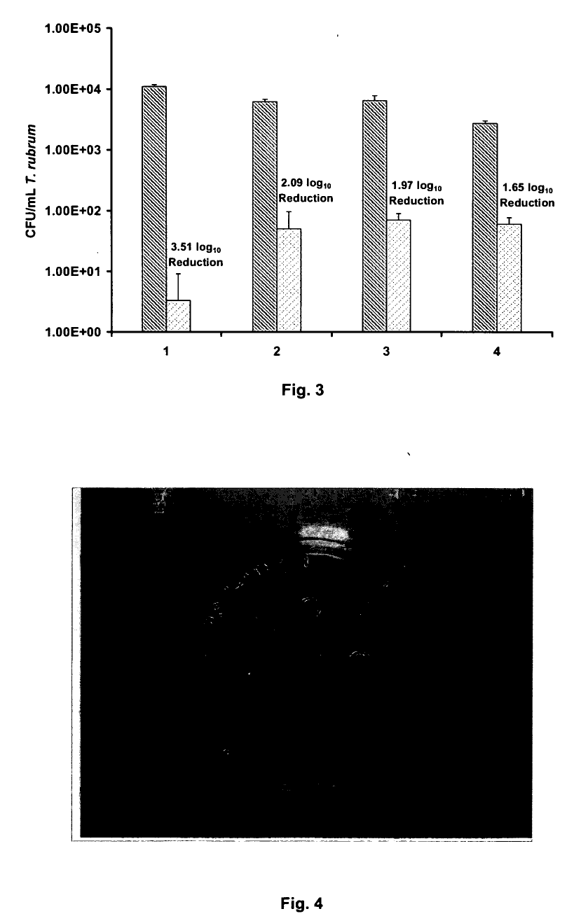

An in vitro experiment was conducted by filling half (48 wells) of a 96 well plate with 100 μl of inoculums in each of the 48 wells and the other half (48 wells) with 100 μl of Periowave® photosensitizer solution, manufactured by Ondine Biopharma Corporation located in Vancouver, Canada containing about 0.01% w/v of methylene blue, in each of the 48 wells. Thereafter, 100 μl of a solution containing Trichophyton rubrum was also added to each of the 96 wells on such plate. Light at 670 nm provided by a 220 mW laser was applied to all of the 96 wells on such plate for 60 seconds. During all of the steps described above, the solutions within the wells were magnetically stirred. The wells containing the inoculums acted as the control group. This experiment was repeated four times and data as shown in FIG. 3 was expressed as viable fungal colonies of Trichophyton rubrum after treatment relative to control. In FIG. 3, the horizontal scale shows the number of experiments (i.e., 4) with each experiment contains two vertical bars. The bars containing the diagonal lines represent the control groups. The bars containing dots represent the photosensitizer treated groups. The vertical scale shows the CFU/ml of Trichophyton rubrum. Data from the four experiments showed that the use of a photosensitizer (e.g., methylene blue) with light provided significant reduction of Trichophyton rubrum. The experiments had a reduction of Trichophyton rubrum from about 1.65 log₁₀ to about 3.51 log₁₀ with an average reduction of about 2.31 log₁₀ (99.5%) compared to the control groups.

### EXAMPLE II

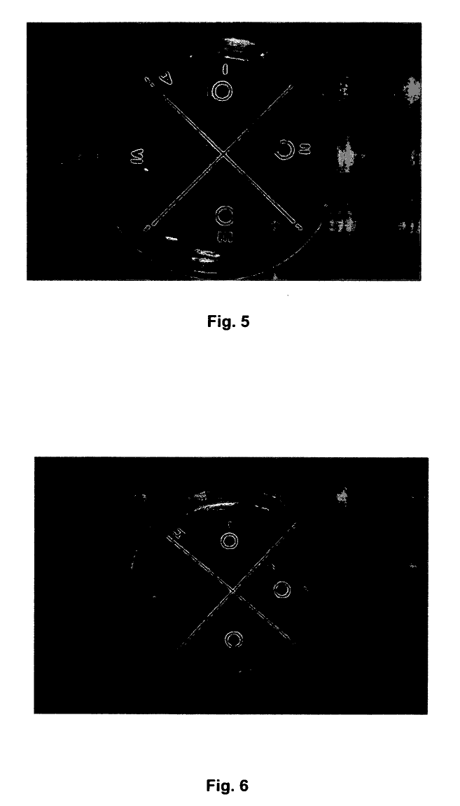

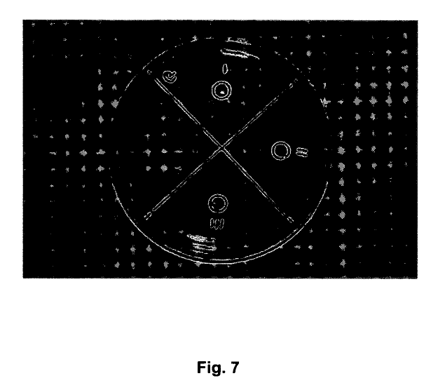

Another in vitro experiment was conducted by dividing each agar plate appropriate for fungal growth into four sections. All four sections were then inoculated with Trichophyton rubrum each in a 7 mm diameter circle. Sections, I, II, and III (starting from top going clockwise) were then exposed to 25 μl of Periowave® photosensitizer solution described in Experiment I and irradiated with a fiber-optically coupled laser at 600 mW for 60 seconds. After the light treatment, the agar plates were incubated at 37° C. to observe fungal growth. Trichophyton rubrum growth in treated Sections I, II, and III was then compared to growth in the untreated control Section IV on each of the agar plates. FIG. 4 showed one of the agar plates after 72 hours of incubation. FIG. 5 showed another one of the agar plates after 96 hours of incubation. FIG. 6 showed another one of the agar plates after 144 hours of incubation. FIG. 7 showed another one of the agra plates after about 9 days of incubation. FIGS. 4-7 showed that Sections I, II, and III had minimal, if any, visible fungal growth as compared to the controls (Section IV) which had visible (white) fungal growth.

### EXAMPLE III

An exemplary protocol for the therapy for treatment of onychomycosis is as follows: Apply Toenail Soft™ or any of the above-discussed nail penetration enhancer to the nail beneath which the locus is situated and to leave it on the nail for 6 to 24 hours. Thereafter, apply the antifungal composition of the present invention containing the photosensitizer to the nail and leave it on the nail for about 15 minutes to about 24 hours. Thereafter, irradiating the locus by applying light to the nail at a wavelength absorbed by the photosensitizer of the antifungal composition so as to destroy microbes at the locus. After the irradiating the locus step, optionally apply a composition comprising the antifungal agent and the pharmaceutically acceptable delivery system wherein the pharmaceutically acceptable delivery system includes the pharmaceutically acceptable nail penetration enhancer; and repeat such application over a predetermined treatment period (e.g., days, weeks, or months).

### EXAMPLE IV

Another exemplary protocol for the therapy for treatment of onychomycosis is as follows: Reduce the thickness (but without actually removing the full structure) of the nail beneath which the locus is situated. This step reduces the nail to at least about half of its thickness or less. This reducing step can be accomplished by (i) controlled laser pulses (e.g., from a femtosecond or other ultrafast excimer laser or the like); and/or (ii) mechanical abrasion such as a file, grit paper, or the like. After the reducing step, apply the antifungal composition to the nail and leave it on the nail for about 15 minutes to about 24 hours. Thereafter, irradiating the locus by applying light to the nail at a wavelength absorbed by the photosensitizer of the antifungal composition so as to destroy microbes at the locus. After the irradiating the locus step, optionally apply a composition comprising the antifungal agent and the pharmaceutically acceptable delivery system wherein the pharmaceutically acceptable delivery system includes the pharmaceutically acceptable nail penetration enhancer; and repeat such application over a predetermined treatment period (e.g., days, weeks, or months).

### EXAMPLE V

Another exemplary protocol for the therapy for treatment of onychomycosis is as follows: Create numerous micro-channels in the nail beneath which the locus is situated using art-disclosed chemical, mechanical, electromagnetic poration means, and a combination thereof. For example, the micro-channels can be created by (i) chemical agents that degrade or solublize the nail matrix in controlled manner; and/or (ii) punctures of the nail by micron-gauge needles, laser or other electromagnetic pulses. The diameter of the micro-channels is preferably from about 1 μm to about 50 μm. After the reducing step, apply the antifungal composition to the nail and leave it on the nail for about 15 minutes to about 24 hours. Thereafter, irradiating the locus by applying light to the nail at a wavelength absorbed by the photosensitizer of the antifungal composition so as to destroy microbes at the locus. After the irradiating the locus step, optionally apply a composition comprising the antifungal agent and the pharmaceutically acceptable delivery system wherein the pharmaceutically acceptable delivery system includes the pharmaceutically acceptable nail penetration enhancer; and repeat such application over a predetermined treatment period (e.g., days, weeks, or months).

### EXAMPLE VI

Same as Examples III, IV, and V above wherein the photosensitizer of the Antifungal composition is methylene blue at a concentration of 0.01% w/v and the length of the light applied is at about 670 nm. The light is applied with a high energy dose (e.g., >20 J/cm2) to the locus through the nail by a laser or LED source. The high energy dose can be applied either by high power for short durations or lower power for long durations.

What is claimed is:

1. An antifungal composition for treating dermatophytic fungal infections comprising:
A photosensitizer;
An effective amount of antifungal agent; and
A pharmaceutically acceptable delivery system.

2. The antifungal composition of claim 1 wherein the pharmaceutically acceptable delivery system includes a pharmaceutically acceptable nail penetration enhancer.

3. The antifungal composition of claim 1 wherein the antifungal agent is comprised of at least one of the following active compounds: (-) menthol, a menthol derivative or analog (e.g., menthone, menthyl salicylate), (-) (1R) menthyl acetate, (-) (1R) menthyl chloride, and menthyloxyacetic acid.

4. The antifungal composition of claim 3 wherein the antifungal agent is further comprised of camphor.

5. The antifungal composition of claim 1 wherein the antifungal agent is selected from the group consisting of: polyenes, allylamines, imidazoles, triazoles, tolnaftate, ciclopirox, morpholines, griseofulvin, and a combination thereof.

6. The antifungal composition of claim 1 wherein the photosensitizer is selected from a group consisting of arianor steel blue, toluidine blue O, crystal violet, methylene blue, methylene blue derivatives, azure blue cert, azure B chloride, azure 2, azure A chloride, azure B tetrafluoroborate, thionin, azure A eosinate, azure B eosinate, azure mix sicc., azure II eosinate, haematoporphyrin HCI, haematoporphyrin ester, aluminium disulphonated phthalocyaninem, porphyrins, pyrroles, tetrapyrrolic compounds, expanded pyrrolic macrocycles, Photofrin® and a combination thereof.

7. The antifungal composition of claim 1 wherein concentration of the photosensitizer is from about 0.0001% to about 10% w/v.

8. The antifungal composition of claim 1 wherein the pharmaceutically acceptable delivery system includes a pharmaceutically acceptable carrier.

9. The antifungal composition of claim 8 wherein the pharmaceutically acceptable carrier is selected from a group consisting of: petroleum hydrocarbon, cetyl alcohol, cetyl palmitate, copolyol, EDTA, glycerin, H2O, imidazole-urea, isopropyl palmitate, methyl paraben, PEG-100 stearate, sodium hydroxide, turpentine, stearic acid, stearyl alcohol, and a combination thereof.

10. The antifungal composition of claim 1 further comprising at least one compound selected from the group consisting of anti-inflammatory agents, buffers, salts, antioxidants, preservatives, and a combination thereof.

11. The antifungal composition of claim 1 wherein the dermatophytic fungal infection is caused by at least one fungus selected from the group consisting of Candida albicans, Trichophyton rubrum, Trichophyton mentagrophytes, and Aspergillus fumigatus, Epidermophyton floccosum, Microsporum canis, and Microsporum gypsum.

12. An antifungal composition for treating dermatophytic fungal infections comprising:
A photosensitizer;
An effective amount of antifungal agent comprising:
(a) menthol in the concentration of from about 2% to about 10% by weight of the antifungal composition;
(b) camphor in the concentration of from about 2% to about 11% by weight of the antifungal composition; and
A pharmaceutically acceptable delivery system comprising carboxymethylcellulose.

13. The antifungal composition of claim 12 wherein the pharmaceutically acceptable delivery system further includes a pharmaceutically acceptable nail penetration enhancer.

14. The antifungal composition of claim 12 wherein the carboxymethylcelluslose is about 2% by weight of the antifungal composition; the menthol is about 4% by weight of the antifungal composition, the camphor is about 2% by weight of the antifungal composition, and the pharmaceutically acceptable delivery system further comprises ethanol in a concentration that is about 30% by weight of the antifungal composition.

15. The antifungal composition of claim 14 wherein the pharmaceutically acceptable delivery system further comprises propylene glycol in a concentration that is about 15% by weight of the antifungal composition.

16. The antifungal composition of claim 14 wherein the pharmaceutically acceptable delivery system further comprises methyl paraben in a concentration that is about 15% by weight of the antifungal composition.

17. The antifungal composition of claim 12 wherein the photosensitizer is methylene blue.

18. The antifungal composition of claim 12 wherein concentration of the photosensitizer is from about 0.0001% to about 10% w/v.

19. The antifungal composition of claim 12 further comprising at least one compound selected from the group consisting of anti-inflammatory agents, buffers, salts, antioxidants, preservatives, and a combination thereof.

20. The antifungal composition of claim 12 wherein the dermatophytic fungal infection is caused by at least one fungus selected from the group consisting of Candida albicans, Trichophyton rubrum, Trichophyton mentagrophytes, and Aspergillus fumigatus, Epidermophyton floccosum, Microsporum canis, and Microsporum gypsum.
